(19) United States
(12) Patent Application Publication
(10) Pub. No.: US 2013/0030513 A1
(43) Pub. Date: Jan. 31, 2013

(54) OSTIAL STENT

(71) Applicant: Richard F. Corrigan, JR., Wheaton, IL (US)

(72) Inventor: Richard F. Corrigan, JR., Wheaton, IL (US)

(21) Appl. No.: 13/190,299

(22) Filed: Jul. 25, 2011

(51) Int. Cl. A61F 2/84

(52) U.S. Cl. 623/1.11

(57) ABSTRACT

An ostial stent for use in improving vessel patency includes a manually-expanding tube section that presents a distal opening of the ostial stent and a pre-shaped self-expanding SMA tube that presents a proximal opening of the ostial stent. The tubes are attached end-to-end to define a passage extending continuously between the openings. The tubes have a generally cylindrical shape in a radially contracted condition so that the tubes can be inserted into the patient. The self-expanding SMA tube is self-expandable from the radially contracted condition to a memory flared condition.

### BACKGROUND

1. Field

The present invention relates generally to stents and methods of accurately placing and securing stents. More specifically, embodiments of the present invention concern an ostial stent with a manually-expanding distal tube section and a self-expanding proximal tube section.

2. Discussion of Prior Art

Stents have long been used to improve the patency of occluded vessels. In one conventional form, balloon-expandable stents are typically made of a relatively strong metal, such as stainless steel. This type of stent is used in vessels where greater radial strength is required. Furthermore, balloon-expandable stents are normally used in areas where the stent is unlikely to be crushed, e.g., by bending/crushing through contact with muscle or other tissues. In another conventional form, self-expanding stents are made of a relatively flexible shape memory alloy material. This type of stent is used where greater flexibility of the stent is required. Conventional stents are sometimes deployed to expand an ostial region. In order to support the ostium, the stent is positioned to extend out into the larger vessel. The protruding portion of the stent is then flared to apply pressure to and support the ostium.

Prior art stents suffer from various undesirable limitations. Conventional stents are not well suited for precise placement in ostial regions of a patient's vascular system so as to conform to the ostial flaring of the larger vessel, particularly in the ostium region between the aorta and renal artery. For instance, balloon-expandable stents are difficult to precisely position in such an ostial region because of artery movement due to beating of the heart and patient breathing. Furthermore, precise positioning is difficult because such stents are slightly radiopaque and, therefore, can be difficult to view during positioning. Even when properly positioned, it may be necessary to flare the proximal end of the stent with the balloon catheter, which can be difficult. Self-expanding shape memory alloy (SMA) stents are deficient in some applications because such stents have less radial strength than balloon-expandable stents. Additionally, SMA stents are less radiopaque than balloon-expandable stents.

### SUMMARY

The following brief summary is provided to indicate the nature of the subject matter disclosed herein. While certain aspects of the present invention are described below, the summary is not intended to limit the scope of the present invention.

Embodiments of the present invention provide an ostial stent system that does not suffer from the problems and limitations of the prior art stents set forth above.

A first aspect of the present invention concerns an ostial stent for simplified and accurate placement at the ostium of a patient's vascular system so as to improve vessel patency in the ostial region. The ostial stent broadly includes a manually-expanding tube and a pre-shaped self-expanding SMA tube. The manually-expanding tube presents a distal opening of the ostial stent. The pre-shaped self-expanding SMA tube presents a proximal opening of the ostial stent, with the tubes being attached end-to-end to define a passage extending continuously between the openings. The tubes have a generally cylindrical shape in a radially contracted condition so that the tubes can be inserted into the patient and the manually-expanding tube is slidable into and out of a vessel of the patient, with the self-expanding SMA tube being selectively positionable at least partially within the ostium. The self-expanding SMA tube is self-expandable from the radially contracted condition to a memory flared condition when heated by exposure to the body temperature of the patient, with the memory flared condition corresponding to a pre-shaped form of the self-expanding SMA tube in which the tube diameter dimension increases proximally.

A second aspect of the present invention concerns a method of implanting a stent at the ostium of a patient's vascular system so as to improve vessel patency in the ostial region. The method broadly includes the steps of positioning the stent into a vessel of the patient so that a pre-shaped self-expanding SMA tube of the stent is located at least partly within the ostium; and permitting the pre-shaped self-expanding SMA tube to self-expand to a flared condition by exposing the stent to the body temperature of the patient.

Other aspects and advantages of the present invention will be apparent from the following detailed description of the preferred embodiments and the accompanying drawing figures.

### BRIEF DESCRIPTION OF THE DRAWING FIGURES

Preferred embodiments of the invention are described in detail below with reference to the attached drawing figures, wherein:

FIG. 1 is a perspective of an ostial stent for use as part of an ostial stent system constructed in accordance with a preferred embodiment of the present invention, with the ostial stent including a self-expanding SMA proximal tube section and a balloon-expandable distal tube section joined end-to-end along a weld line, where the tube sections are made from laser-cut tube material shown schematically, and showing the ostial stent in a radially contracted condition where the tube sections present inner and outer tube diameters that are substantially continuous along the length of the stent;

FIG. 2 is a perspective of the ostial stent shown in FIG. 1, showing the ostial stent in a memory flared condition where the inner and outer tube diameters of the proximal tube section increase in the proximal direction;

FIG. 3 is a schematic view of the ostial stent system inserted in a patient's vascular system, with a fragmentary cross-section of the vascular system taken along a generally longitudinal plane to show the aorta and opposite renal arteries extending laterally to intersect the aorta along respective ostial regions, where one of the ostial regions has deposits therein, with the ostial stent system including the ostial stent, a guide catheter, a guide wire, and a balloon catheter assembly, showing the guide wire extending upwardly into the renal artery, and showing the remaining components of the ostial stent system in a pre-insertion position so that the ostial stent is located in the aorta adjacent the ostial region;

FIG. 4 is a schematic view of the ostial stent system similar to FIG. 3, but showing the ostial stent, guide catheter, and balloon catheter assembly shifted so that the distal end of the guide catheter is located in the ostial region in a stent-insertion position;

FIG. 5 is a schematic view of the ostial stent system similar to FIG. 4, but showing the guide catheter retracted proximally from the stent-insertion position to expose the ostial stent, and showing the ostial stent and balloon catheter assembly shifted distally along the guide wire and into the ostial region, with the proximal tube section being expanded from the radially contracted condition toward a flared condition, where the diameter of the proximal tube section increases in the proximal direction;

FIG. 6 is a fragmentary schematic view of the ostial stent system similar to FIG. 5, but showing the ostial stent and balloon catheter assembly shifted further distally along the guide wire and into the ostial region, with the proximal tube section being further expanded toward the flared condition and engaging the ostial opening by contacting the wall of the aorta so as to restrict further distal advancement of the stent; and

FIG. 7 is a fragmentary schematic view of the ostial stent system similar to FIG. 6, but showing the ostial stent shifted further distally into the ostial region, with the proximal and distal tube sections being expanded to contact and expand the adjacent deposits within the corresponding ostial region.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION OF THE PREFERRED EMBODIMENTS

Turning initially to FIGS. 1, 2, and 3, an ostial stent system 20 is constructed in accordance with a preferred embodiment of the present invention. The ostial stent system 20 is preferably used to implant an ostial stent 22 in an ostial region O of a patient's vascular system and thereby improve vessel patency in the ostial region. As used herein, the term “ostial region” refers to a junction between two vessels. One such junction includes an ostium, which is normally the mouth of the smaller of the two vessels.

As will be discussed further, it has been found that the illustrated system 20 provides for simple and accurate stent implantation in the ostial region. More particularly, the system 20 restricts the operator from advancing the stent too far into the ostium. At the same time, the system 20 signals the operator that the stent has been sufficiently advanced into the ostium. The ostial stent system 20 broadly includes the ostial stent 22, a guide catheter 24, a guide wire 26, and a balloon catheter assembly 28.

The illustrated embodiment has been depicted in use with ostial region O defined by the aorta A and renal arteries R that carry blood from the aorta A to kidneys (not shown). However, the principals of the present invention are equally applicable to other ostial regions within the vascular system V. Returning to the illustrated arrangement, each of the renal arteries R presents a corresponding ostium O between the artery R and aorta A. Generally, the aorta A has a lumen diameter that ranges from about twenty-five (25) millimeters to about thirty-five (35) millimeters. The renal arteries R generally have a lumen diameter that ranges from about four (4) millimeters to about ten (10) millimeters. The inner annular surface of the left ostium O has a plaque deposit D thereon. The deposit D reduces the diameter of the ostium O and undesirably restricts blood flow through the ostium O. Again, the illustrated system 20 is preferably used in the illustrated ostial region O between the aorta A and renal arteries R. However, it is also within the ambit of the present invention to use the system 20 to improve blood flow at other ostial regions in the vascular system V.

Turning to FIGS. 3-5, the ostial stent system 20 is operable to position the stent 22 by initially inserting the guide wire 26 within the patient. The guide wire 26 is a conventional guide wire that extends continuously to a distal end 30. In the usual manner, the guide wire 26 is used to direct the other components of the ostial stent system 20 along the aorta A and into position along the ostial region.

The guide catheter 24 is conventional and preferably includes a continuous catheter tube 32 that presents a guide lumen 34, an outer tube surface 36, and a distal end 38, with the guide lumen 34 extending continuously from a proximal tube end (not shown) to the distal end 38. As will be described, the guide catheter 24 is preferably sized and configured so that the guide lumen 34 can slidably receive the ostial stent 22, guide wire 26, and the balloon catheter assembly 28.

The balloon catheter assembly 28 is also conventional and includes a balloon 40 and a balloon catheter 42. The balloon catheter 42 includes a continuous catheter tube 44 that presents a lumen (not shown), an outer surface, and a distal end 48. The balloon 40 is inflatable and presents proximal and distal ends 50,52, with an outer balloon surface 54 extending between the ends 50,52. The proximal end 50 of the balloon 40 is attached adjacent the distal end 48 of the balloon catheter 42.

The guide catheter 24 and balloon catheter assembly 28 are both slidably received on the guide wire 26, with the balloon catheter assembly 28 being positioned within the guide lumen 34. Thus, the guide catheter 24 and balloon catheter assembly 28 are each slidable along the length of the guide wire 26.

The ostial stent 22 is configured for use in the illustrated vascular system V to improve vessel patency in the ostial region O. While the illustrated ostial stent 22 is preferably used between the aorta A and renal artery R, it is also within the ambit of the present invention to use the ostial stent 22 to improve blood flow at other ostial regions in the vascular system V.

The ostial stent 22 preferably includes a balloon-expandable distal tube section 56 and a self-expanding proximal tube section 58 attached end-to-end. As will be discussed in greater detail, the ostial stent 22 is flared along the axis thereof to distend the ostial region O.

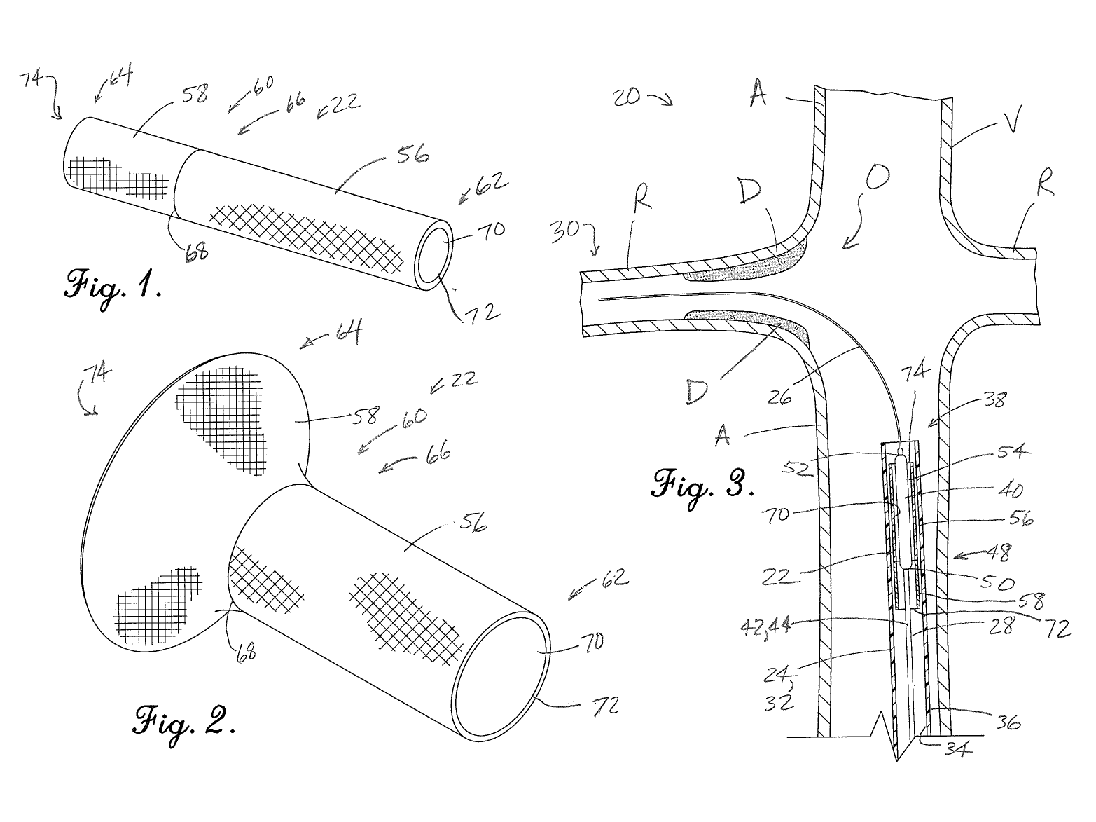

The distal tube section 56 extends continuously between proximal and distal tube ends 60,62 (see FIGS. 1 and 2). Also, the distal tube section 56 presents inner and outer distal tube diameter dimensions. The distal tube section 56 is preferably formed from laser-cut metal tube so that the distal tube section 56 can be manually expanded using a balloon (or another suitable stent-expanding device). However, it is also within the scope of the present invention where the distal tube section 56 is formed from woven metal fabric. The laser-cut metal tube preferably includes stainless steel, but could include other materials, such as chromium-cobalt or a combination thereof, without departing from the scope of the present invention. The distal tube section 56 is preferably shiftable from a radially contracted condition (see FIG. 1) to a radially expanded condition (see FIG. 2). In the radially contracted condition, the outer tube diameter dimension of the tube section 56 is substantially constant along the tube length and preferably ranges from about two (2) millimeters to about four (4) millimeters. In the radially expanded condition, the distal tube section 56 has an enlarged outer tube diameter dimension that preferably ranges from about four (4) millimeters to about ten (10) millimeters. However, it is within the ambit of the present invention where the distal tube section has an outer tube diameter dimension that falls outside of one or both of these ranges.

The proximal tube section 58 extends continuously between proximal and distal tube ends 64,66 and presents inner and outer proximal tube diameter dimensions (see FIGS. 1 and 2). The proximal tube section 58 is also preferably formed of a laser-cut metal tube. However, the principles of the present invention are applicable where the proximal tube section 58 includes a woven metal fabric. The laser-cut metal tube permits expansion and contraction of the proximal tube section 58, as will be discussed.

The metal material of the proximal tube section 58 preferably includes an SMA material. More preferably, the proximal tube section 58 is formed of nickel-titanium (i.e., Nitinol). However, the principles of the present invention are applicable where the proximal tube section 58 includes copper-zinc-aluminum-nickel, copper-aluminum-nickel, or combinations of the referenced SMA materials.

The tube sections 56,58 are initially cut from cylindrical tube stock (not shown). Preferably, the tube sections 56,58 are cut so that the distal tube section 56 presents a distal tube length dimension that is longer than a proximal tube length dimension presented by the proximal tube section 58. However, for some aspects of the present invention, the tube sections 56,58 could be manufactured with alternative tube lengths (e.g., the tube lengths could be the same).

The tube sections are preferably joined end-to-end by attaching the distal end 66 of the proximal tube section 58 to the proximal end 60 of the distal tube section 56. More preferably, the tube sections 56,58 are welded, e.g., by plasma arc welding, to one another along an annular weld line 68 so that the each tube section 56,58 is an integral part of the ostial stent 22. However, the principles of the present invention are equally applicable where other types of welding or joining methods are employed for suitably interconnecting tube sections 56,58. Thus, the tube sections 56,58 cooperatively define a passage 70 that extends continuously between proximal and distal openings 72,74 of the ostial stent 22. In the illustrated embodiment, the overall length of the ostial stent 22 preferably ranges from about ten (10) millimeters to about twenty-five (25) millimeters. However, it is also within the scope of the present invention where the ostial stent 22 has an overall length that falls outside of this range.

The proximal tube section 58 is preferably formed of Nitinol so that the proximal tube section 58 can be sufficiently expanded to at least conform to the shape of the associated vascular structure and, more preferably, even slightly distend the ostial region O. As will be discussed, the proximal tube section 58 preferably self-expands from a radially contracted condition (see FIG. 1) to a memory flared condition (see FIG. 2) when located adjacent the ostial region O. In the radially contracted condition, the outer tube diameter dimension of the proximal tube section 58 is preferably substantially the same as the distal tube section 56. In the memory flared condition, the outer tube diameter dimension of proximal tube section 58 preferably ranges from about four (4) millimeters to about ten (10) millimeters. More specifically, in the memory flared condition, the distal tube end 66 of tube section 58 has an outer diameter dimension that is preferably much smaller than the proximal tube end 64. Preferably, the outer diameter dimension flares continuously outwardly from the distal tube end 66 to the proximal tube end 68 so that the tube section 58 has a sleeve shape that curves along the length thereof. For some aspects of the present invention, the proximal tube section 58 could have alternative dimensions and/or an alternative shape for suitable use of the ostial stent 22.

As discussed above, the tube section 58 is preferably flared outwardly toward the tube end 64 so that the tube end 64 engages the ostium O. This flared shape provides numerous benefits. For instance, the flared stent shape conforms closely to the shape of the vasculature, particularly the ostium O. As a result, the flared end is configured to engage and buttress the ostial wall while restricting inadvertent stent movement into or out of the ostium O. The flared end of stent 22 restricts the operator from advancing the stent too far into the ostium O. Also, through contact with the ostium O, the flared end of stent 22 signals the operator that the stent has been sufficiently advanced into the ostium O. Thus, the flared stent end provides for accurate and simplified stent placement. Consequently, stent implantation procedures can be performed in a shorter period of time. Furthermore, such procedures can reduce the need for implantation of multiple stents in the ostium O due to inaccurate stent placement.

The illustrated proximal tube section 58 is configured for self-expansion in the ostial region O by a process of pre-shaping the proximal tube section 58. For instance, the proximal tube section 58 can be placed in a mold at high temperature and formed into a flared pre-expanded tube shape (not shown) while the Nitinol material is in a high-temperature phase, where the material assumes an Austenite structure. It is also within the ambit of the present invention to use other suitable manufacturing techniques so that the proximal tube section 58 is operable to self-expand when located adjacent the ostial region O. After pre-shaping, the proximal tube section 58 is then permitted to be cooled so as to return to a low-temperature phase, where the material assumes a Martensite structure. In the process of cooling, the tube section 58 self-contracts from the flared pre-expanded tube shape.

The ostial stent 22 is preferably manufactured by initially cutting the tube sections 56,58 to the respective desired lengths from cylindrical tube stock (not shown). The cylindrical tube sections 56,58 are welded in the end-to-end configuration. The proximal tube section 58 of the ostial stent 22 is then preferably pre-shaped, as discussed above, to form the flared pre-expanded tube shape. Again, in the process of cooling to return to the low-temperature phase, the proximal tube section 58 self-contracts from the flared pre-expanded tube shape. After being cooled, the contracted proximal tube section 58 is physically formed to return approximately to the original cylindrical tube shape, with the ostial stent 22 being in the radially contracted condition. This forming step may involve the use of mandrels to roll the proximal tube section back into the original cylindrical tube shape. In this manner, the ostial stent 22 can be subsequently positioned on the balloon and within the guide catheter 24 (see FIG. 3).

Turning to FIGS. 3-7, the system 20 is operable to implant the stent 22 in the ostial region O. Initially, a vascular access site (not shown) is created so that the system 20 can be inserted in the patient. Once the access site is created, the guide wire 26 is inserted and extended into the patient's vascular system V and is positioned along the aorta A so that the distal end 30 can be positioned in the renal artery R. The ostial stent 22 is positioned so that the balloon 40 is received within the ostial stent 22. Preferably, the distal tube section 56 is positioned on the balloon 40, with the proximal tube section 58 extending proximally from adjacent the balloon 40. The ostial stent 22 is also positioned within the guide catheter 24 adjacent the distal end 38 in a covered condition. Thus, the balloon catheter assembly 28, guide catheter 24, and ostial stent 22 can be cooperatively inserted into the patient's vascular system V and passed along the guide wire 26.

The balloon catheter assembly 28, guide catheter 24, and ostial stent 22 are located in a pre-insertion position so that the ostial stent 22 is located in the aorta A adjacent the ostial region O (see FIG. 3). This pre-positioning allows the ostial stent 22 to be conveniently shifted into the ostial region O when the doctor selects a preferred moment for stent insertion into the ostium O.

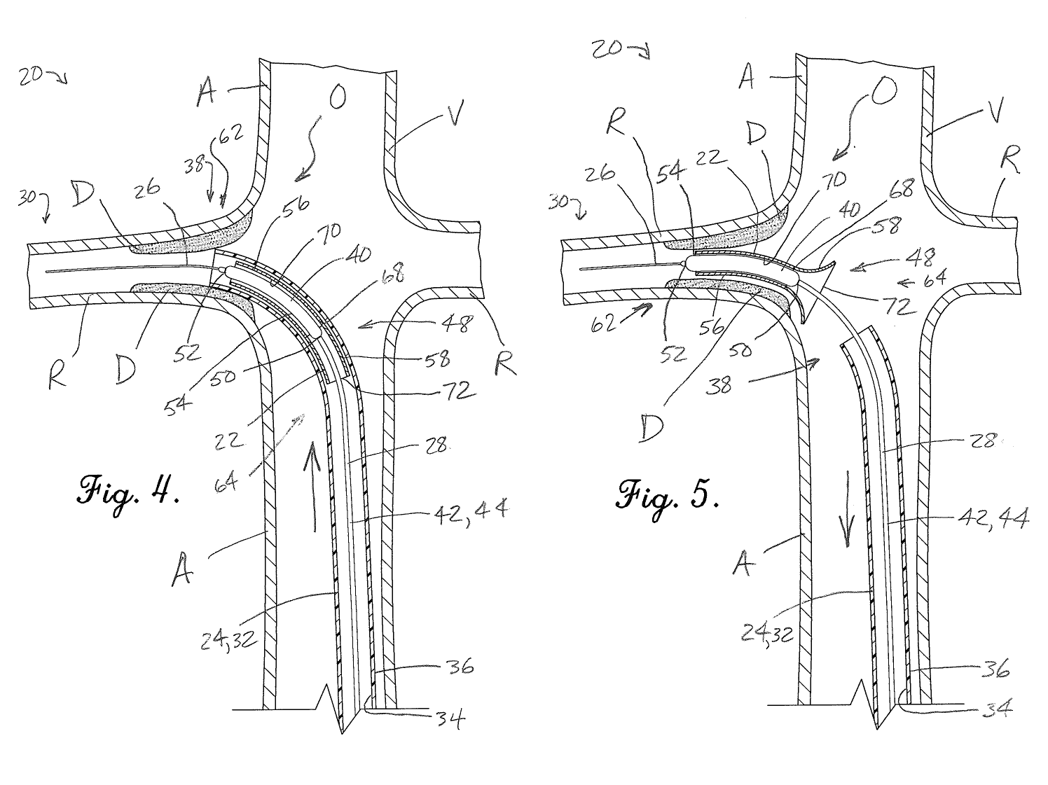

The next step is to shift the balloon catheter assembly 28, guide catheter 24, and ostial stent 22 along the guide wire 26 so that the distal end 38 of the guide catheter 24 is located in the ostial region O in a stent-insertion position (see FIG. 4). The distal end 38 of the illustrated guide catheter 24 is preferably located adjacent the deposits D and within the ostial region O.

From the stent-insertion position, the guide catheter 24 can be retracted to expose the ostial stent 22 (see FIG. 5). As the guide catheter 24 is retracted, the guide catheter 24 no longer restricts self-expansion of the proximal tube section 58. Thus, the proximal tube section 58 begins to self-expand toward the memory flared condition where the diameter of the proximal tube section 58 increases in the proximal direction. This expansion occurs because the SMA material of the proximal tube section 58 is exposed to and heated by the body temperature of the patient. The shape of the proximal tube section 58 in the memory flared condition preferably corresponds to the flared pre-expanded tube shape formed during the pre-shaping process discussed above. It is estimated that the tube section 58 achieves full expansion after a period of exposure within the patient from about twenty-four (24) hours to about forty-eight (48) hours.

With the guide catheter 24 being at least partly retracted, the balloon catheter assembly 28 and ostial stent 22 can be moved distally so that the ostial stent 22 is further inserted into the renal artery R. As the stent 22 is moved distally, the proximal tube section 58 is located within and approaches engagement with the ostial region O. At the same time, the proximal tube section 58 continues to self-expand in diameter toward the memory flared condition. Preferably, the stent 22 is positioned, prior to engagement with the renal artery R, so that a proximal portion of the proximal tube section 58 extends into the aorta A. More preferably, the proximal portion extends a length into the aorta A that ranges from about two (2) millimeters to about three (3) millimeters.

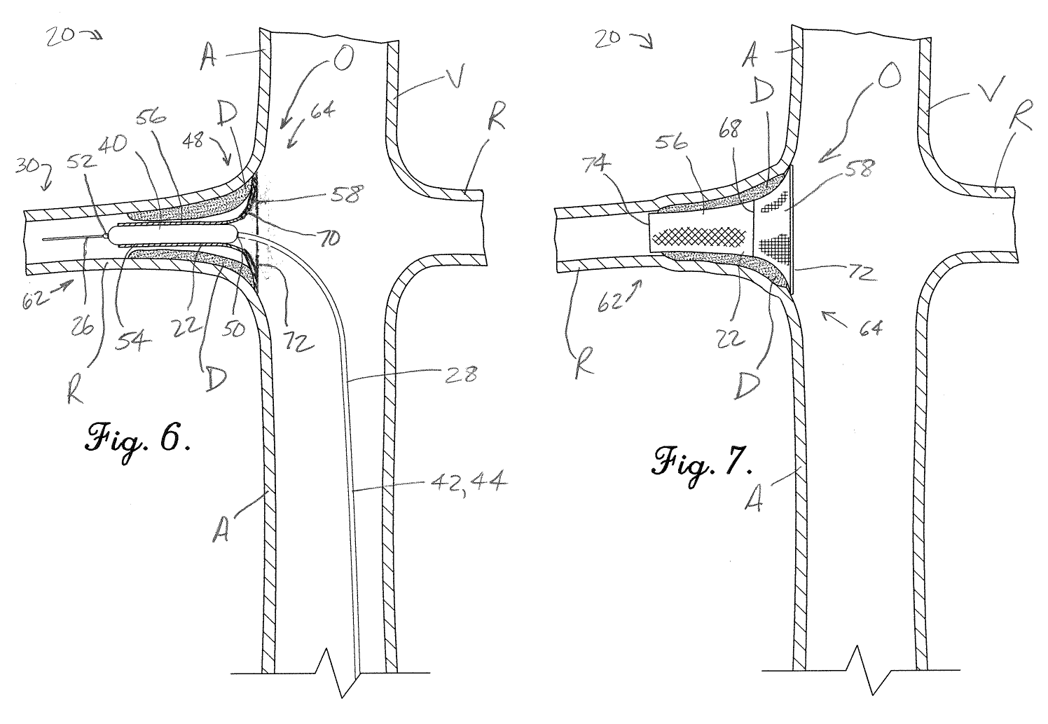

Again, the tube section 58 is preferably flared outwardly toward the tube end 64 so that the tube end 64 engages the ostium O. The flared stent shape conforms closely to the region of the ostial wall. As a result, the flared end is configured to engage and buttress the ostial wall while restricting inadvertent stent movement into or out of the ostium O. In particular, the tube section 58 is preferably flared so as to contact the wall of the aorta A adjacent the proximal tube end 64 (see FIG. 6). In this stent position, engagement of tube section 58 with the aorta A preferably restricts further distal advancement of the stent 22 so that the stent 22 restricts the operator from advancing the stent too far into the ostium O. In engaging the ostium, the flared end of the stent 22 also indicates to the operator that the stent has been sufficiently advanced into the ostium O. Again, these features of the stent permit accurate and simplified stent placement so that stent implantation procedures can be performed in a shorter period of time. Also, such procedures can reduce the need for implantation of multiple stents in the ostium O due to inaccurate stent placement.

The distal tube section 56 is expanded into engagement with the renal artery by manual expansion from a radially contracted condition (see FIG. 6) to a radially distended condition (see FIG. 7). Preferably, the balloon 40 is inflated to apply an expansion pressure within the distal tube section 56 to provide the desired tube expansion. However, it is also within the ambit of the present invention where another mechanism is employed to expand the distal tube section 56. Preferably, the distal tube section 56 has substantially no flaring toward the distal end in the radially distended condition. However, in the expanded condition shown in the drawings (e.g., see FIG. 7), the distal tube section 56 is flared slightly toward the proximal end so as to more closely mimic the vascular shape in which it is positioned.

The illustrated balloon 40 is also preferably used to manually assist with securement of the proximal tube section 58 by manually applying an expansion pressure. Without manual assistance, it is estimated that the proximal tube section 58 expands to about ninety (90) percent of its size when in the flared pre-expanded condition. With the distal tube section 56 secured in the radially distended condition, the balloon 40 can be deflated and shifted proximally to extend along the proximal tube section 58. Once shifted, the balloon 40 can be inflated to urge the proximal tube section 58 into the flared condition.

It is also within the scope of the present invention where the balloon 40 is not used to manually assist with complete expansion of the proximal tube section 58. It is particularly noted that such manual expansion of the stent portion to be located along the flared section of the ostium does not present the same problems as conventional stent designs. With the proximal tube section 58 being already self-expanded, the stent 22 is readily and properly positioned in the ostial region O. Furthermore, with the stent 22 properly positioned, the distal tube section 56 can then be manually expanded to firmly and securely “lock” the stent 22 into place. Then, if necessary, the balloon catheter assembly 28 can be used to facilitate complete expansion of the proximal tube section 58, without concern to the stent location or shape relative to the vessels (as such has already been ensured). In the past, the flared portion of the stent had to be manually formed and located in the ostial region O, which simply was difficult, unpredictable, and time consuming.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventor hereby states his intent to rely on the Doctrine of Equivalents to determine and assess the reasonably fair scope of the present invention as pertains to any apparatus not materially departing from but outside the literal scope of the invention as set forth in the following claims.

What is claimed is:

1. An ostial stent for simplified and accurate placement at the ostium of a patient's vascular system so as to improve vessel patency in the ostial region, said ostial stent comprising:
a manually-expanding tube that presents a distal opening of the ostial stent; and
a pre-shaped self-expanding SMA tube that presents a proximal opening of the ostial stent, with the tubes being attached end-to-end to define a passage extending continuously between the openings,
said tubes having a generally cylindrical shape in a radially contracted condition so that the tubes can be inserted into the patient and the manually-expanding tube is slidable into and out of a vessel of the patient, with the self-expanding SMA tube being selectively positionable at least partially within the ostium,
said self-expanding SMA tube being self-expandable from the radially contracted condition to a memory flared condition when heated by exposure to the body temperature of the patient, with the memory flared condition corresponding to a pre-shaped form of the self-expanding SMA tube in which the tube diameter dimension increases proximally.

2. The ostial stent as claimed in claim 1,
said self-expanding SMA tube being shaped in the high-temperature phase prior to patient insertion to form a flared pre-expanded tube shape, with the SMA tube being operable to contract from the pre-expanded tube shape toward the radially contracted condition when cooled.

3. The ostial stent as claimed in claim 1,
said self-expanding SMA tube including a woven fabric and/or a laser-cut tube SMA material.

4. The ostial stent as claimed in claim 3,
said woven fabric and/or a laser-cut tube SMA material being selected from the group consisting of nickel-titanium, copper-zinc-aluminum-nickel, copper-aluminum-nickel, and combinations thereof.

5. The ostial stent as claimed in claim 1,
said manually-expanding tube being expandable from the radially contracted condition to a radially distended condition by manually applying an expansion pressure within the manually-expanding tube.

6. The ostial stent as claimed in claim 5,
said manually-expanding tube having a generally cylindrical shape in the radially distended condition so as to have substantially no flaring toward the distal end.

7. The ostial stent as claimed in claim 1,
said tubes being welded to each other along an annular path.

8. The ostial stent as claimed in claim 1,
said manually-expanding tube including a woven fabric and/or laser-cut tube material.

9. The ostial stent as claimed in claim 1,
said woven fabric and/or laser-cut tube material being selected from the group consisting of stainless steel, chromium-cobalt, and combination thereof.

10. The ostial stent as claimed in claim 1,
said self-expanding SMA tube having a proximally increasing tube size in the memory flared condition, with a proximal tube diameter dimension being larger than a distal tube diameter dimension.

11. The ostial stent as claimed in claim 1,
said SMA tube presenting an SMA tube length dimension,
said manually-expanding tube presenting a manual tube length dimension, with the manual tube length dimension being longer than the SMA tube length dimension.

12. A method of implanting a stent at the ostium of a patient's vascular system so as to improve vessel patency in the ostial region, said method comprising the steps of:
(a) positioning the stent into a vessel of the patient so that a pre-shaped self-expanding SMA tube of the stent is located at least partly within the ostium; and
(b) permitting the pre-shaped self-expanding SMA tube to self-expand to a flared condition by exposing the stent to the body temperature of the patient.

13. The method as claimed in claim 12,
step (b) including the step of heating the SMA tube so that the SMA tube generally assumes a flared tube shape similar to that formed prior to patient insertion in the high-temperature phase.

14. The method as claimed in claim 13,
step (b) including the step of retracting a guide catheter from a position surrounding the SMA tube so that the guide catheter is prevented from restricting self-expansion of the SMA tube to the flared condition.

15. The method as claimed in claim 12; and
(c) manually assisting with implanting of the SMA tube by manually applying an expansion pressure within the flared SMA tube using a balloon catheter.

16. The method as claimed in claim 13; and
(c) expanding a manually-expanding tube of the stent from a radially contracted condition to a radially distended condition by manually applying an expansion pressure within the manually-expanding tube.

17. The method as claimed in claim 16,
step (c) including the step of using a balloon catheter to apply the expansion pressure.

18. The method as claimed in claim 16; and
(d) manually assisting with implanting of the SMA tube by manually applying an expansion pressure within the flared SMA tube using a balloon catheter.

19. The method as claimed in claim 13,
step (a) including the step of inserting the stent into the patient.

20. The method as claimed in claim 19,
step (a) including the step of creating a vascular access site.

21. The method as claimed in claim 20,
step (a) including the step of inserting a guide wire within the patient and passing a balloon catheter with the stent along the guide wire.

22. The method as claimed in claim 21,
step (a) including the step of positioning the stent within a guide catheter that extends into and out of the patient.

23. The method as claimed in claim 22,
step (b) including the step of retracting the guide catheter from a position surrounding the SMA tube so that the guide catheter is prevented from restricting self-expansion of the SMA tube to the flared condition.

24. The method as claimed in claim 23; and
(c) expanding a manually-expanding tube of the stent from a radially contracted condition to a radially distended condition by manually applying an expansion pressure within the manually-expanding tube.
